(19) United States
(12) Patent Application Publication
(10) Pub. No.: US 2017/0231896 A1
(43) Pub. Date: Aug. 17, 2017

(54) SILICONE ELASTOMER IN OILY PHASE

(71) Applicant: L'OREAL, Paris (FR)

(72) Inventors: Pascal ARNAUD, L'Hay Les Roses (FR); Valerie DIQUE-MOUTON, Cheville Larue (FR)

(73) Assignee: L'OREAL, Paris (FR)

(21) Appl. No.: 15/519,437

(22) PCT Filed: Oct. 16, 2015
(86) PCT No.: PCT/EP2015/073990

§ 371 Date: Apr. 14, 2017

(30) Foreign Application Priority Data
Oct. 17, 2014 (FR) 1460001
Oct. 17, 2014 (FR) 1460007
Oct. 17, 2014 (FR) 1460019

(51) Int. Cl. A61K 8/891; A61Q 1/02; A61K 8/04; A61K 8/29; A61K 8/02

(52) U.S. Cl. CPC A61K 8/891; A61K 8/29; A61K 8/0241; A61K 8/04; A61Q 1/02

(57) ABSTRACT

The present invention relates to a composition, in particular a cosmetic composition, comprising a physiologically acceptable medium containing at least one oily phase, said oily phase comprising: (i) at least one silicone elastomer present within water droplets dispersed in said oily phase; and (ii) at least one polyglycerylated silicone surfactant with an HLB greater than or equal to 8.

The present invention relates to the field of compositions, in particular cosmetic compositions, and more particularly care and/or makeup compositions.

In the cosmetics field, a vast majority of compositions have an oily phase. It is also common practice to thicken this oily phase in order to improve the stability of the corresponding architecture, but also to confer particular properties on the corresponding composition.

It is known to those skilled in the art to introduce, for these purposes, silicone elastomers into oily phases of cosmetic products.

In order to facilitate their use, these silicone elastomers are usually proposed by suppliers in the form of a predispersion in volatile or non-volatile oils, which are introduced into the oily phases of the products during their manufacture.

The compositions described in documents EP 1736 138, EP 1946 799 and U.S. Pat. No. 7,709,013 are precisely products in which the silicone elastomer under consideration has been introduced according to this technique, i.e. in the form of an oily gel.

However, for various reasons, the presence of the oil or oils used to carry the elastomer in the predispersions is not always desired in the final composition. Furthermore, the proportion of these oils in the final composition may occasionally be high, in particular if it is desired to introduce a large amount of elastomers into the composition and if the predispersion contains a small amount thereof.

Some suppliers also propose silicone elastomers in the form of a powder to be dispersed in all or part of the oily phase of the product under consideration, the dispersion thus formed being subsequently used for the manufacture of the expected cosmetic composition. Unfortunately, this dispersion operation can prove to be lengthy and tedious to carry out, with the risk, in the end, of obtaining a non-homogeneous dispersion of the elastomer.

There remains therefore a need for a silicone elastomer in a form dispersible in an oily phase which is homogeneous and does not require the obligatory use of an oily carrier for this elastomer.

It is also known practice to use silicone elastomers carried in the form of an aqueous suspension.

By way of example, a combination of a silicone elastomer coated with a silicone resin with a silicone elastomer carried in the form of an aqueous suspension, giving the product a novel elastic texture and a fresh sensation on application, is known from document FR 2 903 306.

U.S. Pat. No. 5,665,804 describes, for its part, highly concentrated emulsions of silicone elastomers in water, known as “silicone latices” and also cosmetic compositions comprising these emulsions. The concentrated emulsions are then introduced directly into the oily phase of the compositions to gel them without using any surfactant.

However, the use of silicone elastomers in the form of such aqueous suspensions also does not prove to be entirely satisfactory, in particular in terms of homogeneity.

An object of the present invention is therefore to provide a solution which satisfies all the abovementioned requirements.

Unexpectedly and advantageously, the inventors have thus found that it is possible to easily obtain a homogeneous dispersion of a silicone elastomer in an oily phase.

The technology under consideration according to the invention advantageously makes it possible to obtain a homogeneous distribution of the silicone elastomer in any oily phase without the presence of an oily carrier associated with the elastomer.

Thus, according to a first of its aspects, the present invention relates to a composition, in particular a cosmetic composition, comprising a physiologically acceptable medium containing at least one oily phase, said oily phase comprising:

(i) at least one silicone elastomer present within water droplets dispersed in said oily phase; and

(ii) at least one polyglycerylated silicone surfactant with an HLB greater than or equal to 8.

A composition according to the invention is advantageously a liquid composition, the continuous phase of which is oily and in which the silicone elastomer is dispersed within water droplets. Advantageously, the composition according to the invention is present in the form of an emulsion comprising an external oily phase, which contains the polyglycerylated silicone surfactant according to the invention.

More specifically, the inventors have noted that the choice of a polyglycerylated silicone surfactant with an HLB greater than or equal to 8 makes it possible to advantageously obtain a composition of which the continuous oily phase is thickened via the stabilized and homogeneous dispersion, in said phase, of an aqueous suspension of a silicone elastomer.

This result is all the more surprising since this family of surfactants is generally considered for stabilizing emulsions of oil-in-water type and which therefore have an architecture that is very different from that under consideration according to the invention which favours a continuous oily phase.

In the context of the present invention, the compositions are therefore prepared by bringing together an oily phase containing this polyglycerylated silicone surfactant and an aqueous phase containing in particular the silicone elastomer and which can in particular be represented by the aqueous suspension of this elastomer. This aqueous phase is advantageously dispersed, with stirring, in the oily phase already containing this polyglycerylated silicone surfactant.

The invention therefore makes it possible to simply obtain a composition formed from a continuous oily phase in which the aqueous phase is homogeneously dispersed in a stabilized manner, in the form of water droplets carrying in particular the silicone elastomer. As emerges from the examples hereinafter, the compositions according to the invention advantageously provide the desired properties through the oils which make up the oily phase thereof which is continuous, while at the same time being efficiently and uniformly thickened.

As emerges from the examples hereinafter, a composition according to the invention has an improved stability, in particular in terms of homogeneity. It is also comfortable to apply.

The inventors have noted that an aqueous suspension of a silicone elastomer can effectively perform the role of a carrier for one or more water-soluble compounds within an oily phase, provided that a surfactant in accordance with the invention is combined with this aqueous suspension of elastomer. The aqueous phase carrying the elastomer and the water-soluble compound(s) proves to be homogeneously dispersed in a stabilized manner within the oily phase by virtue of the simultaneous presence of at least one polyglycerylated silicone surfactant with an HLB greater than or equal to 8.

According to one particular embodiment, a composition according to the invention comprises at least one water-soluble compound, said compound being carried within the water droplets of the elastomer dispersion (i).

This or these water-soluble compound(s) is (are) therefore advantageously in a form solubilized in the oily phase and, in this respect, capable of providing an immediate effect on application when the water droplets containing it (them) rupture.

As emerges from the examples hereinafter, the proposed combination according to the invention makes it possible to obtain a homogeneous and stable distribution of a water-soluble compound in any oily phase, without requiring the presence of a large amount of water, the immediate effect of which would be to affect the properties desired through the use of an oily phase.

The inventors have also discovered that the composition of the invention is conducive to the homogeneous and stable dispersion of particles of nacre type.

According to another particular embodiment, a composition according to the invention comprises reflective particles of average size less than or equal to 30 μm, and made up of a natural or synthetic mica-based substrate coated with a single layer or several superimposed layers, the weight proportion of a TiO₂ layer, if present, remaining less than that of the mica.

As emerges from the examples hereinafter, the undesirable sedimentation phenomenon which gives the formulation a two-phase appearance does not manifest itself according to the invention. The compositions according to the invention thus advantageously have a single-phase and shimmering appearance in their entirety.

A subject of the invention is also a process for making up and/or caring for a keratin material, and in particular the skin, comprising a step of applying a composition according to the invention.

The term “keratin material” covers in particular keratin fibres such as the hair and the eyelashes.

The term “skin” is intended to denote all of the skin of the body, including the lips, and preferably the skin of the face, the neck and the neckline.

The invention also relates to a process for preparing a cosmetic composition according to the invention, comprising at least one step of introducing at least one silicone elastomer in aqueous suspension as defined above into an oily phase and at least one step of introducing at least one surfactant as defined above into said oily phase. These steps may be simultaneous or successive.

According to a preferred embodiment, the step of introducing a silicone elastomer in aqueous suspension into the oily phase is performed after the step of introducing at least one surfactant in accordance with the invention into the oily phase.

According to one of its variants, the invention also relates to a process for preparing a cosmetic composition according to the invention, comprising at least:

a) a step of introducing at least one silicone elastomer in aqueous suspension into an oily phase, and

b) a step of introducing at least one polyglycerylated silicone surfactant with an HLB greater than or equal to 8 into said oily phase,

the two introducing steps being simultaneous or consecutive, said aqueous suspension of silicone elastomer also containing at least one water-soluble compound.

Preferably, the silicone elastomer and the surfactant as defined above are introduced into the same oily phase.

According to another of its variants, the invention also relates to a process for preparing a cosmetic composition according to the invention, comprising at least:

a) a step of introducing at least one silicone elastomer in aqueous suspension into an oily phase,

b) a step of introducing at least one polyglycerylated silicone surfactant with an HLB greater than or equal to 8 into said oily phase, and

c) a step of introducing the reflective particles according to the invention into the oily phase containing said elastomer(s) and surfactant(s),

it being possible for steps (a) and (b) to be simultaneous or successive.

Preferably, the silicone elastomer and the surfactant as defined above are introduced into the same oily phase.

Silicone Elastomers in Aqueous Suspension

The silicone elastomers under consideration according to the invention are present in the compositions within water droplets dispersed in the oily phase of this composition.

The term “elastomer” is intended to mean a supple, deformable material with viscoelastic properties and in particular the consistency of a sponge or a supple sphere. This elastomer is formed from polymer chains of high molecular weight, the mobility of which is limited by a uniform network of crosslinking points.

The silicone elastomers used in the composition according to the invention may be in the form of particles of at least one partially or totally crosslinked organopolysiloxane elastomer having a three-dimensional structure. They are particulate. They may be chosen in particular from the crosslinked polymers described in U.S. Pat. No. 5,928,660 from Dow Corning.

According to that patent, the crosslinked organopolysiloxane elastomer is obtained via addition and crosslinking reaction, in the presence of a catalyst in particular of platinum type, of at least:

(a) an organopolysiloxane (i) containing at least two vinyl groups in the α-ω position of the silicone chain per molecule; and

(b) an organosiloxane (ii) containing at least one hydrogen atom linked to a silicon atom per molecule.

The aqueous dispersion may in particular be obtained as follows:

(a) mixing of an organopolysiloxane (i) containing at least two vinyl groups in the α-ω position of the silicone chain per molecule and of an organosiloxane (ii) containing at least one hydrogen atom linked to a silicon atom per molecule;

(b) adding a catalyst, in particular of platinum type;

(c) adding an aqueous phase containing an emulsifier to form an emulsion;

(d) polymerization of the organopolysiloxane (i) and of the organosiloxane (ii) in emulsion in the presence of a catalyst.

In particular, the organopolysiloxane (i) is chosen from α,ω-dimethylvinylpolydimethylsiloxanes.

Reference may be made to L'OREAL document FR 2 800 605 for a detailed description of the process for manufacturing these aqueous suspensions of silicone elastomers.

The organopolysiloxane elastomer particles have a size ranging from 0.1 to 500 microns, and better still from 3 to 200 microns. These particles may be spherical, flat or amorphous, and preferably spherical in shape.

These organopolysiloxane elastomer particles are stably dispersed in water by means of one or more non-ionic, cationic or anionic emulsifiers with an HLB greater than or equal to 8.

The proportion of emulsifiers in the aqueous suspension preferably ranges from 0.1 to 20 parts by weight per 100 parts by weight of the aqueous suspension of organopolysiloxane elastomer and better still from 0.5 to 10 parts by weight.

Thus, the silicone elastomers, contained within water droplets dispersed in the oily phase of a composition according to the invention, are present therein in a form combined with one or more surfactants.

These surfactants are different from the polyglycerylated silicone surfactant with an HLB greater than or equal to 8 required in parallel according to the invention.

According to one particular embodiment, the silicone elastomer, and its surfactants, are combined in an elastomer/surfactant(s) weight ratio, expressed by dry weight, ranging from 30 to 1000.

The expression “forms combined with” is intended to mean that the silicone elastomer and its combined surfactant(s) are not present separately in the oily phase of the composition of the invention, but are intimately combined with one another.

This mode of combination between said silicone elastomer and its surfactant(s) is in particular like that obtained following the emulsion polymerization process generally considered for forming a silicone elastomer. This process requires the presence of a surfactant in order to carry out the polymerization reaction and this surfactant remains present in a combined form in the silicone elastomer particles thus obtained.

Such combinations of silicone elastomer with one or more surfactant(s) also exist in commercially available forms of silicone elastomers which are in the form of aqueous suspensions or in a dry form as indicated in the INCI names of the commercial products more particularly identified hereinafter.

The surfactant combined with the silicone elastomer in the dispersed water droplets is preferably non-ionic.

The preferred non-ionic surfactants are oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; and oxyalkylenated (oxyethylenated and/or oxypropylenated) fatty alcohol ethers.

By way of non-ionic surfactants which may be combined with the silicone elastomer, mention may also quite particularly be made of the surfactants sold under the names Tergitol® TMN-6, Tergitol® 15S40, Tergitol® 15S3, Tergitol® 15S5 and Tergitol® 15S7 from Union Carbide Corporation (Danbury, Conn.), and Brij 30 and Brij 35 from ICI Chemicals (Wilmington, Del.); Triton® X405 from Röhm & Haas (Philadelphia, Pa.); Makon 10 from Stepan Company (Northfield, Ill.); Ethomid O/17 from Akzo Inc. (Chicago, Ill.) and Pluronic® F38 from BASF Corporation (Parsippany, N.J.).

The non-ionic surfactants are preferably C₁₀-C₁₈ fatty polyoxyethers, i.e. a polymer of polyethylene glycol (PEG) and of C₁₀-C₁₈ fatty acids.

It is preferably a question of a polymer of polyethylene glycol (PEG) and of C₁₀-C₁₈ fatty acids comprising from 4 to 12 EO units.

Examples of non-ionic surfactants that are quite particularly suitable are: laureth-4®, laureth-12® and C₁₂-C₁₄ pareth-12®.

The aqueous suspensions of organopolysiloxane particles suitable for the preparation of the composition of the invention are, for example, those sold under the names BY 29-119, BY 29-122, BY 29-129 and DC 9509 by the company Dow Corning, the US INCI name of which is Dimethicone/Vinyl Dimethicone Crosspolymer and C12-C14 pareth-12.

These suspensions comprise about 63% by weight of organopolysiloxane elastomer particles (and thus about 63% active material) relative to their total weight.

Mention may also be made of the reference Gransil LTX sold by the company Grant Industries, the INCI name of which is Cyclopentasiloxane (and) polysilicone-11 (and) water (and) laureth-4 and in which the content of organopolysiloxane elastomer particles is between 14% and 20%.

Finally, mention may be made of the reference Gransil EP-9, also sold by the company Grant Industries, the INCI name of which is Polysilicone-11 (and) water (and) laureth-12 (and) phenoxyethanol. (and) ethylhexylglycerine and in which the content of non-volatile matter is between 59% and 62%.

The silicone elastomer is advantageously present in the composition of the invention in an active material amount ranging from 0.1% to 30% by weight, preferably from 1% to 25% by weight and even more preferentially from 5% to 20% by weight, relative to the total weight of the composition.

Polyglycerolated Silicone Surfactant

As previously stated, the silicone elastomer as defined above is present in the compositions according to the invention together with at least one polyglycerylated silicone surfactant with an HLB greater than or equal to 8.

It is recalled that the term “HLB (Hydrophilic-Lipophilic Balance)” is intended to mean the equilibrium between the size and the strength of the hydrophilic group and the size and the strength of the lipophilic group of the surfactant under consideration.

This HLB value according to Griffin is defined in J. Soc. Cosm. Chem. 1954 (volume 5), pages 249-256.

Advantageously, this polyglycerylated silicone surfactant with an HLB greater than or equal to 8 corresponds to formula (I) below:

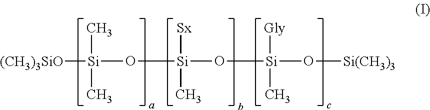

in which:

a ranges from 1 to 1.4; b ranges from 0.02 to 0.04; and c ranges from 0.02 to 0.04,

Sx is the radical —O(CH₃)₂SiO—Si(CH₃)₃; and

Gly is a hydrocarbon-based radical chosen from glycerylated derivatives.

Such compounds are in particular described in documents EP 1 213 316 and EP 1 640 040.

More specifically, the glycerylated derivatives may be compounds having formulae as follows:

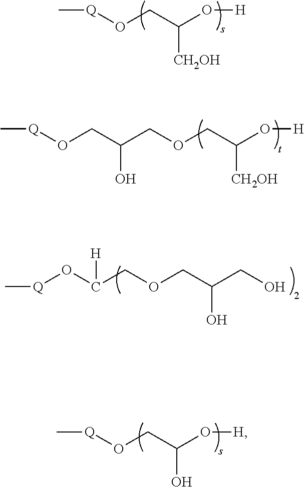

in which

Q is a divalent radical chosen from —(CH₂)₂— and —(CH₂)₃—, and

s and t are integers included in the range of from 1 to 20, in particular from 1 to 15, in particular from 1 to 10, and more particularly from 1 to 5.

Preferably, Gly can be represented by the radical —C₃H₆O[CH₂—CH(OH)CH₂O]ₙH with n ranging from 1 to 5.

According to one preferred variant of the invention, the polyglycerylated silicone surfactant is a dimethicone of polyglyceryl-3 disiloxane, of formula (I) in which:

Sx is the radical —O(CH₃)₂SiO—Si(CH₃)₃,

Gly is the radical —C₃H₆O[CH₂—CH(OH)CH₂O]ₙH with n ranging from 1 to 5, and

a ranges from 1 to 1.4; b ranges from 0.02 to 0.04; and c ranges from 0.02 to 0.04.

Such a compound is in particular sold by the company Shin-Etsu under the reference KF6100®, the HLB of which is evaluated at from 8.0 to 10.0.

A composition according to the invention may advantageously comprise from 1% to 15% by weight, preferably from 4% to 10% by weight and even more preferentially from 5% to 8% by weight of polyglycerylated silicone surfactant(s) with an HLB greater than or equal to 8, relative to the total weight of the composition.

According to the invention, the weight ratio between the silicone elastomer (in active material) and the polyglycerylated silicone surfactant may advantageously have a value which ranges from 0.01 to 6, preferably from 0.1 to 5, and even more preferentially from 0.5 to 4.

Oily Phase

As emerges from the aforementioned, the present invention relates more particularly to the cosmetic compositions which have an oily phase, and more particularly a continuous oily phase.

This oily phase can be of very varied nature and its composition is generally adapted to the keratin target to which the corresponding cosmetic composition is dedicated, namely the lips, the skin or the head of hair, and to the desired purpose, namely makeup and/or care.

For the purposes of the invention, an oily phase comprises at least one oil.

The term “oil” is intended to mean any fatty substance that is in liquid form at ambient temperature and atmospheric pressure.

An oily phase that is suitable for preparing the cosmetic compositions according to the invention may comprise hydrocarbon-based oils, silicone oils, fluoro oils or non-fluoro oils, or mixtures thereof.

They may be of animal, plant, mineral or synthetic origin.

For the purposes of the present invention, the term “silicone oil” is intended to mean an oil comprising at least one silicon atom, and in particular at least one Si—O group.

The term “fluoro oil” is intended to mean an oil comprising at least one fluorine atom.

The term “hydrocarbon-based oil” is intended to mean an oil containing mainly hydrogen and carbon atoms.

The oils may optionally comprise oxygen, nitrogen, sulfur and/or phosphorus atoms, for example in the form of hydroxyl or acid radicals.

The oils may be volatile or non-volatile.

For the purposes of the invention, the term “volatile oil” is intended to mean any oil that is capable of evaporating on contact with the skin in less than one hour, at ambient temperature and atmospheric pressure. The volatile oil is a volatile cosmetic compound, which is liquid at ambient temperature, in particular having a nonzero vapour pressure, at ambient temperature and atmospheric pressure, in particular having a vapour pressure ranging from 0.13 Pa to 40 000 Pa (10⁻³ to 300 mmHg), in particular ranging from. 1.3 Pa to 13 000 Pa (0.01 to 100 mmHg) and more particularly ranging from 1.3 Pa to 1300 Pa (0.01 to 10 mmHg).

For the purposes of the present invention, the term “non-volatile oil” is intended to mean an oil with a vapour pressure of less than 0.13 Pa.

Volatile Oils

The volatile oils may be hydrocarbon-based oils or silicone oils.

Among the volatile hydrocarbon-based oils containing from 8 to 16 carbon atoms, mention may be made in particular of branched C₈-C₁₆ alkanes, such as C₈-C₁₆ isoalkanes (also known as isoparaffins), isododecane, isodecane, isohexadecane and, for example, the oils sold under the trade names Isopar or Permetyl, branched C₈-C₁₆ esters, such as isohexyl neopentanoate, and mixtures thereof. Preferably, the volatile hydrocarbon-based oil is chosen from volatile hydrocarbon-based oils containing from 8 to 16 carbon atoms, and mixtures thereof, in particular from isododecane, isodecane and isohexadecane, and is in particular isohexadecane.

Mention may also be made of volatile linear alkanes comprising from 8 to 16 carbon atoms, in particular from 10 to 15 carbon atoms and more particularly from 11 to 13 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, mixtures of n-undecane (C₁₁) and of n-tridecane (C₁₃) obtained in Examples 1 and 2 of application WO 2008/155 059 from the company Cognis, and mixtures thereof.

Volatile silicone oils that may be mentioned include linear volatile silicone oils such as hexamethyldisiloxane, octamethyltrisiloxane, decamethyltetrasiloxane, dodecamethylpentasiloxane, tetradecamethylhexasiloxane and hexadecamethylheptasiloxane.

Volatile cyclic silicone oils that may be mentioned include hexamethylcyclotrisiloxane, octamethylcyclotetrasiloxane, decamethylcyclopentasiloxane and dodecamethylcyclohexasiloxane.

Non-Volatile Oils

The non-volatile oils may be chosen in particular from non-volatile hydrocarbon-based, fluoro and/or silicone oils.

Non-volatile hydrocarbon-based oils that may in particular be mentioned include:

hydrocarbon-based oils of animal origin, for instance perhydrosqualene,

hydrocarbon-based oils of plant origin, such as liquid triglycerides of fatty acids containing from 4 to 10 carbon atoms, for instance heptanoic or octanoic acid triglycerides, or alternatively, for example, sunflower oil, maize oil, soya bean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol 810, 812 and 818 by the company Dynamit Nobel, jojoba oil and rhea butter oil;

synthetic ethers containing from 10 to 40 carbon atoms, such as dicapryl ether,

synthetic esters, such as the oils of formula R₁COOR₂, in which R₁ represents a linear or branched fatty acid residue comprising from 1 to 40 carbon atoms and R₂ represents a hydrocarbon-based chain, which is in particular branched, containing from 1 to 40 carbon atoms, on condition that R₁+R₂≧10. The esters may be chosen in particular from fatty acid alcohol esters, for instance cetostearyl octanoate, isopropyl alcohol esters such as isopropyl myristate or isopropyl palmitate, ethyl palmitate, 2-ethylhexyl palmitate, isopropyl stearate, octyl stearate, 2-octyldodecyl stearate, 2-octyldodecyl erucate, isostearyl isostearate, hydroxylated esters, such as isostearyl lactate or octyl hydroxystearate, alkyl or polyalkyl ricinoleates, hexyl laurate, fatty alcohol heptanoates, octanoates and decanoates, neopentanoic acid esters, such as isodecyl neopentanoate or isotridecyl neopentanoate, octyldodecyl neopentanoate, and isononanoic acid esters, such as isononyl isononanoate or isotridecyl isononanoate,

polyol esters, such as propylene glycol dioctanoate, neopentyl glycol diheptanoate, and diethylene glycol diisononanoate, and pentaerythritol esters, such as dipentaerythritol tetrahydroxystearate/tetraisostearate,

fatty alcohols that are liquid at ambient temperature, with a branched and/or unsaturated carbon-based chain containing from 12 to 26 carbon atoms, for instance 2-octyldodecanol, isostearyl alcohol and oleyl alcohol,

C₁₂-C₂₂ higher fatty acids, such as oleic acid, linoleic acid, linolenic acid, and mixtures thereof.

Non-volatile silicone oils that may in particular be mentioned include:

non-phenyl silicone oils, for instance dimethicones with a viscosity greater than or equal to 10 cSt, and alkyl dimethicones such as caprylyl methicone, and

phenyl silicone oils, for instance phenyl trimethicones, phenyl dimethicones, phenyltrimethylsiloxydiphenyl siloxanes, diphenyl dimethicones, diphenylmethyldiphenyltrisiloxanes, 2-phenylethyl trimethylsiloxysilicates and trimethylpentaphenyltrisiloxane, and mixtures thereof; and also mixtures of these various oils.

Advantageously, in a composition according to the invention, the oily phase(s) may comprise, or even consist of, one or more silicone oil(s).

According to one embodiment, decamethyltetrasiloxane, dodecamethylpentasiloxane, cyclohexasiloxane and isododecane are the preferential oils.

A composition according to the invention may advantageously comprise from 5% to 95% by weight, better still from 5% to 40% by weight and preferably from 7% to 35% by weight of oil(s) relative to the total weight of said composition.

Preferably, in the composition according to the invention, the proportion of the oily phase(s) may be from 3% to 98% by weight, preferably from 10% to 90% by weight and preferably from 20% to 80% by weight, relative to the total weight of the composition.

Preferably, in a composition according to the invention, the proportion of the oily phase(s) may comprise from 35% to 95% by weight, advantageously from 5% to 95% by weight, preferably from 5% to 40% by weight and preferably from 7% to 35% by weight of oil(s), relative to the total weight of the composition.

The oily phase may also contain other fatty substances.

The other constituents that may be present in the oily phase are, for example, fatty acids, hydrocarbon-based or non-hydrocarbon-based waxes, and silicone resins.

These constituents may be chosen in a varied manner by those skilled in the art so as to prepare a composition having the desired properties, for example in terms of consistency or texture.

Aqueous Phase

A composition according to the invention comprises an aqueous phase totally or partially made up of the water carrying the silicone elastomer and which is dispersed in the oily phase therefore forming the continuous phase of the compositions according to the invention.

This aqueous phase, comprising at least the water of the aqueous dispersion of the elastomer required according to the invention, can thus be supplemented by any water-soluble supplementary compound. It should be noted that these supplementary additives are advantageously carried by this aqueous phase. In other words, they are introduced into the composition by dispersion of the aqueous phase containing them in the oily phase required according to the invention.

These additives may be water, a floral water such as cornflower water and/or a mineral water such as Vittel water, Lucas water or La Roche Posay water and/or a spring water.

The water provided by the aqueous suspension of silicone elastomer and that optionally added to this suspension may be present in a composition according to the invention in a content ranging from 0.02% to 50% by weight, preferably ranging from 0.25% to 40% by weight and more preferentially ranging from 1% to 30% by weight relative to the total weight of the composition.

Water-Soluble Compounds

According to one particular embodiment, a composition according to the invention also comprises at least one water-soluble compound, said compound being carried within the water droplets of the elastomer dispersion (i).

The term “water-soluble compound” is intended to mean a compound which has a solubility in water, measured at 25° C., at least equal to 0.01 g/l (production of a macroscopically isotropic, transparent, coloured or colourless solution).

As explained above, this or these compound(s) is (are) carried within the oily phase, by means of the water droplets of the aqueous suspension of an elastomer, these droplets being homogeneously dispersed in a stabilized manner in the oily phase by virtue of the simultaneous presence in this phase of a polyglycerylated silicone surfactant with an HLB greater than or equal to 8.

The introduction of this or these water-soluble compound(s), even in a significant amount, within the aqueous phase of the aqueous suspension of elastomer does not affect the dispersion thereof, in the form of stabilized droplets in the oily phase.

This or these water-soluble compound(s) is (are) therefore advantageously in a form solubilized in the oily phase and, in this respect, capable of providing an immediate effect on application when the water droplets containing it (them) rupture.

Moreover, when the or one of the water-soluble compounds carried is, for example, a water-soluble dye, its presence within the water droplets homogeneously dispersed in the oily phase can confer thereon an original and attractive aesthetic effect.

As emerges from the examples hereinafter, the proposed combination according to the invention makes it possible to obtain a homogeneous and stable distribution of a water-soluble compound in any oily phase, without requiring the presence of a large amount of water, the immediate effect of which would be to affect the properties desired through the use of an oily phase.

The water-soluble compounds which may be included in the composition according to the present invention can in particular be chosen from compounds intended to confer a cosmetic property, such as a provision of colour, a modification of texture, solar protection, an improvement in wear property, skin moisturization, etc.

A non-limiting illustration of the water-soluble compounds which may be used in a composition according to the invention may thus be C₁-C₈ monoalcohols such as ethanol, polyols, polymers, active agents, screening agents and dyes, and mixtures thereof.

Among the polyols, mention may be made of glycols comprising from 2 to 8 carbon atoms, such as 1,2-propanediol, 1,3-propanediol, 1,2-butanediol, 1,2-pentanediol, isoprene glycol, hexylene glycol, dipropylene glycol and caprylyl glycol, polyethylene glycols, and polyhydric alcohols comprising from 2 to 8 carbon atoms, such as glycerol.

Among the water-soluble polymers of synthetic origin, mention may be made, as examples, of ethylene oxide homopolymers and copolymers; polyvinyl alcohols; vinylpyrrolidone homopolymers and copolymers; vinylcaprolactam homopolymers and copolymers; polyvinyl methyl ether homopolymers and copolymers; neutral acrylic homopolymers and copolymers; C₁-C₂-alkyl-celluloses and derivatives thereof; C₁-C₃-alkyl-guar or C₁-C₃-hydroxyalkyl-guar. Such polymers are in particular described in detail in l'Oreal patent FR 2 811 564.

Among the water-soluble polymers of natural origin, mention may be made of optionally modified polymers of natural origin, such as gum arabic, guar gum, xanthan derivatives, karaya gum, alginates and carrageenans.

Among the water-soluble active agents, mention may be made of alpha-hydroxy acids, such as lactic acid or glycolic acid, beta-hydroxy acids, such as salicylic acid, ascorbic acid (vitamin C) and its biologically compatible salts, constituents which have a tensioning effect, hydroxylated polyacids, sugars, such as rhamnose and sucrose and its derivatives, urea and its derivatives, such as hydroxyethylurea, adenosine, amino acids, oligopeptides, water-soluble plant extracts, peptide or protein hydrolysates, hyaluronic acid and its salts, mucopolysaccharides, vitamins B2, B3, B6, B9 and H, panthenol, allantoin, glycyrrhetic acid, kojic acid, flavonoids, anti-ageing active agents, such as hydroxypropyltetrahydropyrantriol, jasmonic acid derivatives such as sodium 4 hydroxyjasmonate, and lightening active agents such as phenylethyl resorcinol.

Among the water-soluble screening agents, mention may be made of the screening agent of which the INCI name is terephthalydene dicamphor sulfonic acid, sold under the reference Mexoryl SX by the company Chimex, and the screening agent of which the INCI name is phenylbenzimidazole sulfonic acid, sold under the reference Eusolex 232 by the company Merck.

Among the water-soluble dyes, mention may be made of natural or synthetic water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper-containing chlorophylline, methylene blue, anthocyanins (enocyanin, black carrot, hibiscus or elder), caramel and riboflavin.

The water-soluble compounds may be present in the composition of the invention in an active material amount ranging from 0.0001% to 20% by weight, preferably from 0.01% to 10% by weight and even more preferentially from 0.1% to 5% by weight, relative to the total weight of the composition.

Additives

In a known manner, the cosmetic composition of the invention may also contain adjuvants that are common in the cosmetics field, such as gelling agents, preserving agents, fragrances, fillers, UV-screening agents, bactericides, odour absorbers, colorants, plant extracts, salts, antioxidants, basic agents or acidic agents, and mixtures thereof.

The amounts of these various adjuvants are those conventionally used in the field under consideration, for example from 0.0001% to 20% of the total weight of the composition. Depending on their nature, these adjuvants can be introduced into the oily phase, or into the aqueous phase forming a composition according to the invention.

Colorants

The composition in accordance with the invention may also comprise at least one colorant.

According to one embodiment, a composition according to the invention may comprise at least one colorant chosen, for example, from organic colorants and inorganic colorants, such as pigments, liposoluble dyes, nacres, and materials with a specific optical effect, and mixtures thereof.

The term “pigments” should be understood as meaning white or coloured, inorganic (mineral) or organic particles, which are intended to colour the resulting film.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

The liposoluble dyes are, for example, Sudan red, DC Red 17, DC Green 6, β-carotene, soya bean oil, Sudan brown, DC Yellow 11, DC Violet 2, DC Orange 5 and quinoline yellow.

The colorants may be present in a proportion of from 0.0001% to 40% by weight, in particular from 0.01% to 25% by weight or even from 0.1% to 15% by weight, relative to the total weight of the composition.

According to another embodiment, a composition according to the invention is free of colorant, and may constitute a makeup base or a care product for the skin or the hair.

Reflective Particles

According to one particular embodiment, a composition according to the invention also comprises reflective particles of average size less than or equal to 30 μM, and made up of a natural or synthetic mica-based substrate coated with a single layer or several superimposed layers, the weight proportion of a TiO₂ layer, if present, remaining less than that of the mica.

Unexpectedly, the inventors have in fact discovered that the use, for thickening purposes, of an aqueous suspension of a silicone elastomer together with a surfactant as defined above makes it possible to obtain a thickened oily phase conducive to the homogeneous and stable dispersion of particles of nacre type.

This formulation with particles in suspension is also devoid of adverse side effect regarding the aesthetic and comfort properties expected for the resulting cosmetic formulation. Thus, the compositions in accordance with the invention prove to be advantageously devoid of tacky and mattified effect. Furthermore, they provide an attractive visual effect from the viewpoint of the homogeneous dispersion of nacres. As emerges from the examples hereinafter, the undesirable sedimentation phenomenon which gives the formulation a two-phase appearance does not manifest itself according to the invention. The compositions according to the invention thus advantageously have a single-phase and shimmering appearance in their entirety.

The term “reflective particles” is intended to mean, in the general sense, particles of which the size, the structure, in particular the thickness of the layer(s) of which they are constituted and their physical and chemical natures, and the surface appearance, enable them to reflect incident light with a sufficient intensity to be able to create at the surface of the composition claimed, when the latter is applied to the support to be made up, points of brightness that are visible to the naked eye, i.e. more luminous points that contrast with their surroundings by appearing to shine.

The reflective particles may disrupt the visual perception of the curvature of the made-up support, by tending to prevent lasting visual focusing, the points of brightness being liable to appear or disappear at random when the made-up support and the observer are moving.

The reflective particles used must be compatible with cosmetic use and must be able to remain in the composition, and in particular to not dissolve, or in any case to not dissolve entirely, therein.

Thus, the reflective particles advantageously have a size that is compatible with the manifestation of specular reflection of visible light (400-700 nm), of sufficient intensity to create a point of brightness. This size is liable to vary according to the chemical nature of the particles, their shape and their power for specular reflection of visible light.

In the context of the present invention, the reflective particles have an average size of less than or equal to 30 μm, preferably ranging from 1 to 25 μm, and preferably from 2 to 20 μm.

The average size of the reflective particles according to the invention corresponds to their apparent diameter since they are comparable, during the measurement, to spheres. The average size is characterized by the D50 which corresponds to the average particle size of half the population. The particle size measurement can be carried out in the following way:

0.1 g of reflective particles is introduced into 12 to 14 g of isopropyl myristate, in a flask. The sample is stirred until a complete dispersion is obtained, then the particle size of the nacres in this medium is measured using the Mastersizer 2000 from the company Malvern.

Generally, the reflective particles are multilayer particles.

More specifically, they are made up of a natural or synthetic mica-based substrate, coated with a single layer or several superimposed layers, at least one of the layers being a layer of metal oxide, chosen for example from titanium oxide, in particular TiO₂, iron oxide, in particular Fe₂O₃, tin oxide, chromium oxide, barium sulfate and the following materials: MgF₂, CrF₃, ZnS, ZnSe, SiO₂, Al₂O₃, MgO, Y₂O₃, SeO₃, HfO₂, ZrO₂, CeO₂, Nb₂O₅, Ta₂O₅, MoS₂ and mixtures or alloys thereof.

According to the invention, the substrate can also be coated with bismuth oxychloride, ultramarine blue, Prussian blue, manganese violet and/or cochineal carmine.

Such particles are also known as nacres. The nacres may have a yellow, pink, red, bronze, orangey, brown, gold and/or coppery tint.

In the context of the present invention, they are advantageously particles made up of a natural or synthetic mica substrate, partially or totally coated with one or more layers, at least one of the layers being a layer of metal oxide chosen from titanium dioxide, iron, tin and chromium oxides, and silica and alumina.

In the case where the reflective particles have at least one layer of titanium dioxide, the weight proportion of mica is greater than that of titanium dioxide.

Non-limiting examples of nacres that may be mentioned in particular, alone or as a mixture, include nacres comprising the following elements:

mica-titanium oxide, for example sold under the trade reference Timiron Supersilk MP 1005 by the company Merck and the reference EXP 1373-088C Sun Shine soft shine red by the company Sun Chemical;

mica-iron oxide, for example sold under the trade reference Pearlescent Pigment Prestige Soft Bronze from Sudarshan;

mica-titanium oxide-iron oxide, for example sold under the trade reference Colorona oriental beige by the company Merck;

mica-titanium oxide-brown iron oxide-tin oxide, for example sold under the trade reference Syncrystal Ivory by the company Eckart.

A composition of the invention may thus comprise from 0.1% to 30% by weight, preferably from 0.5% to 20% by weight and even more preferentially from. 1% to 10% by weight of reflective particles, relative to the total weight of the composition, advantageously carried in its oily phase.

According to this variant of the invention, the weight ratio between the silicone elastomer (in active material) and the reflective particles may advantageously have a value which ranges from 0.2 to 100, preferably from 1 to 50, more preferentially from 3 to 20 and even more preferentially from 5 to 10.

Cosmetic Active Agents

Those skilled in the art may also add one or more additional cosmetic active agent(s) according to the effect desired on the keratin materials.

Advantageously, these active agents are carried in the oily phase of a composition according to the invention. The cosmetic active agents may be present in the composition according to the invention in a content ranging from 0.001% to 20% by weight, preferably from 0.01% to 10% by weight, even more preferentially from 0.5% to 5% by weight, relative to the total weight of the composition.

Galenical Form

The compositions according to the invention comprise a physiologically acceptable medium, i.e. a non-toxic medium which can be applied to human skin, and which has a pleasant appearance, odour and feel.

In particular, it will be a cosmetic composition for making up and/or caring for the skin or the hair, and more particularly the face.

More specifically, the compositions according to the invention may be makeup products, preferably of the type such as foundations, makeup bases, face powders, eyeshadows, concealer products or blushers, or alternatively a makeup product and/or a product for colouring the skin, the hair or the eyelashes, and in particular foundations. Preferably, the composition according to the invention is a makeup base or a foundation.

Likewise, the compositions according to the invention may be products for caring for the skin, the hair or the eyelashes, and more particularly the skin, and preferably a composition for protecting, treating or caring for the face, for example a day cream, night cream, antisun composition, protective or care body milk or after-sun milk, and more preferably a facial care composition.

In addition, the compositions used according to the invention may be more or less fluid and may have the appearance of a white or coloured cream, an ointment, a milk or a paste. Preferentially, the compositions according to the invention are fluid.

The invention will now be illustrated by the non-limiting examples that follow.

### EXAMPLES

### Examples 1 to 5

### Influence of the Nature of the Surfactant

Examples 1 to 5 make it possible to show the influence of the structure of the surfactant on the dispersibility of the aqueous suspension of silicone elastomer (BY 29-119 from Dow Corning) in an oily phase.

| | | % by weight |
|---|---|---|
| A | Surfactant | 10.00 |
| | Isohexadecane | 27.00 |
| | Isododecane | 19.60 |
| | Dodecamethylpentasiloxane | 28.00 |
| B | Aqueous suspension of organopolysiloxane | 15.40 (9.70 |
| | elastomer (63% elastomer) sold under the | AM) |
| | reference BY 29-119 by the company Dow Corning | |
| | TOTAL | 100% |

Procedure

The constituents of phase A are weighed out in the main beaker and are stirred with a Moritz blender (1100 rpm) while maintaining at ambient temperature.

Phase B is then added while stirring with the Moritz blender (3000 rpm) for 10 minutes until homogenized.

Results

| | | Appearance of |
|---|---|---|
| | | the dispersion |
| Example | Silicone surfactant | (×10 microscope) |
| Example 1 | Polyglyceryl-3 Disiloxane | Homogeneous dispersion |
| (Invention) | Dimethicone sold under the | of the elastomer by |
| | reference KF6100 by the | microscope |
| | company Shin-Etsu | |
| Example 2 | Polyglyceryl-3 Polydimethyl- | Heterogeneous dispersion |
| (Comparative) | siloxyethyl Dimethicone sold | of the elastomer by |
| | under the reference KF 6104 | microscope (presence of |
| | by Shin-Etsu | residual lumps) |
| Example 3 | Lauryl Polyglyceryl-3 | Heterogeneous dispersion |
| (Comparative) | Polydimethylsiloxyethyl | of the elastomer by |
| | Dimethicone sold under | microscope (presence of |
| | the reference KF 6105 by | residual lumps) |
| | Shin-Etsu | |
| Example 4 | Dimethicone Polyglycerin-3 | Heterogeneous dispersion |
| (Comparative) | Crosspolymer sold under the | of the elastomer by |
| | reference KSG 710 by Shin- | microscope (presence of |
| | Etsu | residual lumps) |
| Example 5 | Bis PEG/PPG-16/16 PEG/ | Heterogeneous dispersion |
| (Comparative) | PPG-16/16 Dimethicone | of the elastomer by |
| | sold under the reference | microscope (presence of |
| | Abil Care 85 by the company | residual lumps) |
| | Evonik Goldschmidt | |

### Examples 6 to 9

### Influence of the Nature of the Silicone Elastomer

Examples 6 to 9 make it possible to show the influence of the nature of the silicone elastomer on the homogeneity of the composition and its viscosity.

| | | Example 6 | Example 7 | Example 8 | Example 9 |
|---|---|---|---|---|---|
| | | (Invention) | (Invention) | (Comparative) | (Comparative) |
| | | % by weight | % by weight | % by weight | % by weight |
| A | Polyglyceryl-3 Disiloxane | 6.00 | 6.00 | 6.00 | 6.00 |
| | Dimethicone sold under the | | | | |
| | reference KF6100 by the | | | | |
| | company Shin-Etsu | | | | |
| | Dodecamethylpentasiloxane | 66.00 | 64.60 | 66.00 | 54.24 |
| B | Aqueous suspension of | 28.00 (17.64% | — | — | — |
| | organopolysiloxane | AM + | | | |
| | elastomer (63% elastomer) | 10.18% Water) | | | |
| | sold under the reference BY | | | | |
| | 29-119 by the company | | | | |
| | Dow Corning | | | | |
| | Aqueous suspension of | — | 29.40 (17.64% | — | — |
| | organopolysiloxane | | AM + | | |
| | elastomer (60% elastomer) | | 11.17% Water) | | |
| | sold under the reference EP- | | | | |
| | 9 by the company Grant | | | | |
| | Industries | | | | |
| | Vinyl dimethicone/ | — | — | 17.64 | — |
| | Methicone Silsesquioxane | | | | |
| | crosspolymer sold under the | | | | |
| | reference KSP 100 by the | | | | |
| | company Shin-Etsu | | | | |
| | Dimethicone/Vinyl | — | — | — | 29.40 (17.64% |
| | dimethicone crosspolymer + | | | | AM + |
| | Dimethicone 6 cSt (60/40) | | | | 11.76% |
| | sold under the reference | | | | Dimethicone) |
| | KSG 6 by the company | | | | |
| | Shin-Etsu | | | | |
| C | Water | — | — | 10.36 | 10.36 |
| | TOTAL | 100% | 100% | 100% | 100% |
| | Homogeneity of the | Homogeneous | Homogeneous | Homogeneous | Non- |
| | composition | | | | homogeneous |

Procedure

The constituents of phase A are weighed out in the main beaker and are stirred with a Moritz blender (1100 rpm) while maintaining at ambient temperature.

Next, phase B is added at ambient temperature, while stirring using a Moritz blender (3000 rpm) for 10 minutes until homogenized.

Phase C is then added, with continued stirring using a Moritz blender (3000 rpm) for 5 minutes.

Results

| | | Viscosity |
|---|---|---|
| Example | Silicone elastomer | (mPa · s) |
| Example 6 | Aqueous suspension of organopoly- | 1020 |
| (Invention) | siloxane elastomer (63% elastomer) | |
| | sold under the reference BY 29-119 | |
| | by the company Dow Corning | |
| Example 7 | Aqueous suspension of organopoly- | 1060 |
| (Invention) | siloxane elastomer (60% elastomer) | |
| | sold under the reference EP-9 by | |
| | the company Grant Industries | |
| Example 8 | Vinyl dimethicone/Methicone | 100 |
| (Comparative) | Silsesquioxane crosspolymer sold | |
| | under the reference KSP 100 by | |
| | the company Shin-Etsu | |
| Example 9 | Dimethicone/Vinyl dimethicone | Not |
| (Comparative) | crosspolymer + Dimethicone 6 cSt | measurable |
| | (60/40) sold under the reference KSG | since not |
| | 6 by the company Shin-Etsu | homogeneous |

Procedure for Measuring Viscosity

The viscosity is measured after 24 hours of storage in a room kept at a constant temperature of 20° C.

A Rheomat 200 from Lamy is used as viscometer.

The principle of the measurement is based on the determination of the couple required to overcome the resistance of the fluid using an immersed element (measuring spindle or body) rotating at a constant speed.

The spindle is chosen in such a way that the measurement is always between 10 and 90 du (whole number of deviation units). In the context of this invention, spindle 2 or spindle 3 is used (spindles ranging from 1 (most fluid) to 5 (thickest)).

The product is introduced into a bucket suitable for the chosen spindle and kept at a temperature of 25° C. by virtue of a thermostatic water bath.

The programming of the apparatus is the following:

Mode: manual

Measuring system: 75 (fixed parameter regardless of the spindle used and programmed by the company Lamy)

Rotational speed: 200 rpm

The reading is carried out after 10 minutes of rotation of the spindle.

### Example 10

### Foundation

| | | % by weight |
|---|---|---|
| A | Polyglyceryl-3 Disiloxane Dimethicone | 6.00 |
| | sold under the reference KF6100 by the | |
| | company Shin-Etsu | |
| | Isododecane | 11.00 |
| | Dodecamethylpentasiloxane | 40.71 |
| B | Aqueous suspension of organopolysiloxane | 22.00 (13.86 |
| | elastomer (63% elastomer) sold under the | AM) |
| | reference BY 29-119 by the company Dow Corning | |
| C | Dodecamethylpentasiloxane | 6.00 |
| | Titanium dioxide coated with aluminium stearoyl | 12.47 |
| | glutamate | |
| | Yellow iron oxide coated with aluminium stearoyl | 1.26 |
| | glutamate | |
| | Red iron oxide coated with aluminium stearoyl | 0.40 |
| | glutamate | |
| | Black iron oxide coated with aluminium stearoyl | 0.16 |
| | glutamate | |
| | TOTAL | 100% |

Procedure

The constituents of phase A are weighed out in the main beaker and are stirred with a Moritz blender (1000 rpm) for 15 minutes while maintaining at ambient temperature.

Next, phase B is added to the main beaker while stirring using a Moritz blender (2500 rpm) for 10 minutes until homogenized.

Phase C is prepared separately by milling three times in a three-roll mill the mixture of pigments and of dodecamethylpentasiloxane.

Next, this phase C is added to the main beaker while stirring using a Moritz blender (3500 rpm) for 15 minutes at ambient temperature.

### Examples 11 and 12

Examples 11 and 12 illustrate the possibility of introducing respectively a polyol and a water-soluble polymer into the aqueous phase of a suspension of a silicone elastomer and of then introducing this suspension into the oily phase.

| | | Example 11 | Example 12 |
|---|---|---|---|
| | | (Invention) | (Invention) |
| | | % by weight | % by weight |
| A | Polyglyceryl-3 Disiloxane | 6.00 | 6.00 |
| | Dimethicone sold under the | | |
| | reference KF6100 by the | | |
| | company Shin-Etsu | | |
| | Dodecamethylpentasiloxane | 64.00 | 65.90 |
| B | Aqueous suspension of | 28.00 (17.64% | 28.00 (17.64% |
| | organopolysiloxane | AM + | AM + |
| | elastomer (63% elastomer) | 10.18% Water) | 10.18% Water) |
| | sold under the reference | | |
| | BY 29-119 by the company | | |
| | Dow Corning | | |
| | Propane-1,3 diol sold under | 2.00 | — |
| | the reference Zemea propane | | |
| | diol by the company Dupont | | |
| | Tate and Lyle Bio Products | | |
| | Polyvinylpyrrolidone sold | — | 0.10 |
| | under the reference PVP K | | |
| | 90 by the company ISP | | |
| | Homogeneity of the composition | Homogeneous | Homogeneous |

Procedure

The constituents of phase A are weighed out in the main beaker and are stirred with a Moritz blender (1100 rpm) while maintaining at ambient temperature.

Phase B is prepared separately by dissolving the water-soluble compound in the aqueous phase of the silicone elastomer while stirring with a Rayneri mixer (500 rpm) for 5 min.

Next, phase B is added to phase A at ambient temperature, while stirring using a Moritz blender (3000 rpm) for 10 minutes until homogenized.

### Examples 13 to 16

Examples 13 to 16 illustrate the possibility of introducing water-soluble compounds into the aqueous phase of a suspension of a silicone elastomer and of then introducing this suspension into the oily phase.

| | | Example 13 | Example 14 | Example 15 | Example 16 |
|---|---|---|---|---|---|
| | | (Invention) | (Invention) | (Invention) | (Invention) |
| | | % by weight | % by weight | % by weight | % by weight |
| A | Polyglyceryl-3 Disiloxane Dimethicone | 6.00 | 6.00 | 6.00 | 6.00 |
| | sold under the reference KF6100 by the | | | | |
| | company Shin-Etsu | | | | |
| | Dodecamethylpentasiloxane | 58.41 | 57.00 | 59.50 | 64.00 |
| B | Aqueous suspension of | 28.00 (17.64% | 28.00 (17.64% | 28.00 (17.64% | 28.00 (17.64% |
| | organopolysiloxane elastomer (63% | AM + | AM + | AM + | AM + |
| | elastomer) sold under the reference BY | 10.18% Water) | 10.18% Water) | 10.18% Water) | 10.18% Water) |
| | 29-119 by the company Dow Corning | | | | |
| | Hydroxyethylurea sold under the | 7.59 (3% | — | — | — |
| | reference Hydrovance by the company | AM) | | | |
| | Akzo Nobel | | | | |
| | Hydroxypropyltetrahydropyrantriol sold | — | 9.00 (3.15% | — | — |
| | under the reference Mexoryl SBB by the | | AM) | | |
| | company Chimex | | | | |
| | Sodium tetrahydrojasmonate sold under | — | — | 6.50 (2.08% | — |
| | the reference Mexoryl SBO by the | | | AM) | |
| | company Chimex | | | | |
| | Niacinamide (Vitamin B3) | — | — | — | 2.00 |
| | Homogeneity of the composition | Homogeneous | Homogeneous | Homogeneous | Homogeneous |

Procedure

The compositions of Examples 13 to 16 are prepared according to the procedure described for Examples 11 and 12.

### Example 17

Example 17 illustrates the possibility of introducing a water-soluble screening agent into the aqueous phase of a suspension of a silicone elastomer and of then introducing this suspension into the oily phase.

| | | Example 17 |
|---|---|---|
| | | (Invention) |
| | | % by weight |
| A | Polyglyceryl-3 Disiloxane Dimethicone | 6.00 |
| | sold under the reference KF6100 by the | |
| | company Shin-Etsu | |
| | Dodecamethylpentasiloxane | 56.79 |
| B | Aqueous suspension of organopolysiloxane | 28.00 (17.64% AM + |
| | elastomer (63% elastomer) sold under the | 10.18% Water) |
| | reference BY 29-119 by the company Dow | |
| | Corning | |
| | Terephthalylidene dicamphor sulfonic | 7.00 (2.31% AM) |
| | acid sold under the reference Mexoryl | |
| | SX by the company Chimex, | |
| | Triethanolamine | 2.21 |
| | Homogeneity of the composition | Homogeneous |

Procedure

The constituents of phase A are weighed out in the main beaker and are stirred with a Moritz blender (1100 rpm) while maintaining at ambient temperature.

Phase B is prepared separately by dissolving the water-soluble screening agent in the aqueous phase of the silicone elastomer while magnetically stirring for 5 minutes, and then it is neutralized with triethanolamine while continuing the magnetic stirring for 5 minutes.

Next, phase B is added to phase A at ambient temperature, while stirring using a Moritz blender (3000 rpm) for 10 minutes until homogenized.

### Example 18

Example 18 illustrates the possibility of introducing a water-soluble dye into the aqueous phase of a suspension of a silicone elastomer and of then introducing this suspension into the oily phase.

| | | Example 18 |
|---|---|---|
| | | (Invention) |
| | | % by weight |
| A | Polyglyceryl-3 Disiloxane Dimethicone | 6.00 |
| | sold under the reference KF6100 by the | |
| | company Shin-Etsu | |
| | Dodecamethylpentasiloxane | 67.70 |
| B | Aqueous suspension of organopolysiloxane | 26.00 (16.38% AM + |
| | elastomer (63% elastomer) sold under the | 9.45% Water) |
| | reference BY 29-119 by the company Dow | |
| | Corning | |
| | Red 4 | 0.30 |
| | Homogeneity of the composition | Homogeneous |

Procedure

The composition of Example 18 is prepared according to the procedure described for Examples 11 and 12.

### Examples 19 and 20

### Influence of the Nature of the Substrate of the Nacre

Examples 19 and 20 make it possible to show the influence of the nature of the substrate of the nacre which according to the invention should be mica.

| | | % by weight |
|---|---|---|
| A | Polyglyceryl-3 Disiloxane Dimethicone | 6.00 |
| | sold under the reference KF6100 by the | |
| | company Shin-Etsu | |
| | Dodecamethylpentasiloxane | 66.00 |
| B | Aqueous suspension of organopolysiloxane | 26.00 (16.38 AM) |
| | elastomer (63% elastomer) sold under the | |
| | reference BY 29-119 by the company Dow | |
| | Corning | |
| C | Nacre | 2.00 |
| | TOTAL | 100 |

Procedure:

The constituents of phase A are weighed out in the main beaker and are stirred with a Moritz blender (1100 rpm) while maintaining at ambient temperature.

Phase B is then added while stirring with the Moritz blender (3000 rpm) for 10 minutes until homogenized.

Next, phase C is added at ambient temperature, by stirring using a Moritz blender (3100 rpm) until homogenized.

Results

Appearance after 1 month of stability at ambient temperature 37, 45 and 4° C.:

| | | | Average | |
|---|---|---|---|---|
| Examples | Nacres | Structure | size (μm) | Appearance |
| Example 19 | Mica-brown iron oxide sold | Mica/Iron ox. | 8 | No |
| (invention) | under the reference Pearlescent | (43/57) | | sedimentation of |
| | Pigment Prestige soft bronze by | | | the nacre. |
| | the company Sudarshan | | | Homogeneous |
| | Chemical | | | dispersion. |
| Example 20 | Silica- titanium oxide-tin oxide | SiO₂/TiO₂/SnO₂ | 20 | Totally |
| (Comparative) | sold under the reference Xirona | (88/10/2) | | unacceptable |
| | Magic Mauve by the company | | | appearance at |
| | Merck | | | 45° C.: |
| | | | | Sedimentation |
| | | | | of the nacre. |

### Examples 21 to 25

### Influence of the Size and of the Content of Mica of the Nacre

The comparison of Examples 21 to 24 makes it possible to show the influence of the average size of the nacre which is preferably between 1 and 30 μm.

The comparison of Examples 21 and 25 makes it possible to show the influence of the content of mica of the nacre which is preferably greater than that of the titanium dioxide.

| | | % by weight |
|---|---|---|
| A | Polyglyceryl-3 Disiloxane Dimethicone sold | 6.00 |
| | under the reference KF6100 by the company | |
| | Shin-Etsu | |
| | Dodecamethylpentasiloxane | 66.00 |
| B | Aqueous suspension of organopolysiloxane | 26.00 (16.38 AM) |
| | elastomer (63% elastomer) sold under the | |
| | reference BY 29-119 by the company Dow | |
| | Corning | |
| C | Nacre | 2.00 |
| | TOTAL | 100 |

Procedure:

These compositions are prepared according to the procedure described in detail for Examples 19 and 20.

Results

Appearance after 1 month of stability at ambient temperature 37, 45 and 4° C.:

| Examples | Nacres | Structure | Size (μm) | Appearance |
|---|---|---|---|---|
| Example 21 | Mica-titanium oxide sold | Mica/TiO₂ | 8 | No sedimentation of |
| (invention) | under the reference Timiron | (60/40) | | the nacre. Dispersion |
| | Supersilk MP 1005 by the | | | Homogeneous. |
| | company Merck | | | |
| Example 22 | Synthetic mica | Synthetic | 15 | No sedimentation of |
| (invention) | (fluorphlogopite)-Titanium | mica/TiO₂/ | | the nacre. Dispersion |
| | oxide-Brown iron oxide sold | Iron ox./SnO₂ | | Homogeneous. |
| | under the reference Syncrystal | (73/24/2/1) | | |
| | Ivory by the company Eckart | | | |
| Example 23 | Synthetic mica | Synthetic | 45 | Totally unacceptable |
| (Comparative) | (fluorphlogopite)-brown iron | mica/Iron ox. | | appearance: |
| | oxide sold under the reference | (54.5/45.5) | | Sedimentation of the |
| | Sunshine Glitter Russet by | | | nacre at 37° C. and |
| | the company Sun Chemical | | | increase in viscosity at |
| | | | | 45° C. |
| Example 24 | Mica-brown iron oxide sold | Mica/Iron ox. | 80 | Totally unacceptable |
| (Comparative) | under the reference Colorona | (83/17) | | appearance at 45° C.: |
| | glitter bronze by | | | Sedimentation of the |
| | the company Merck | | | nacre and increase in |
| | | | | viscosity. |
| Example 25 | Mica-titanium oxide sold | Mica/TiO₂/SnO₂ | 12 | Totally unacceptable |
| (Comparative) | under the reference Timiron | (31/68/1) | | appearance at 45° C.: |
| | silk green by the company | | | beginning of |
| | Merck | | | agglomeration of the |
| | | | | nacre. |

### Example 26

### Nacreous Oily Illuminator

| | | % by weight |
|---|---|---|
| A | Polyglyceryl-3 Disiloxane Dimethicone | 5.00 |
| | sold under the reference KF6100 by the | |
| | company Shin-Etsu | |
| | Polydimethylsiloxane sold under the reference | 10.00 |
| | Xiameter PMX-200 Silicone fluid 5 cSt by the | |
| | company Dow Corning | |
| | Cyclohexasiloxane | 27.00 |
| | Isododecane | 10.00 |
| | Dodecamethylpentasiloxane | 20.00 |
| B | Aqueous suspension of organopolysiloxane | 26.00 (16.38 AM) |
| | elastomer (63% elastomer) sold under the | |
| | reference BY 29-119 by the company Dow | |
| | Corning | |
| C | Mica-titanium oxide-brown iron oxide | 2.00 |
| | (49/32/19) sold under the reference | |
| | Colorona oriental beige by the company Merck | |
| | TOTAL | 100 |

Procedure:

This composition is prepared by reproducing the preparation process described for Examples 19 and 20.

### Example 27

### Nacreous Oily Illuminator

| | | % by weight |
|---|---|---|
| A | Polyglyceryl-3 Disiloxane Dimethicone | 6.00 |
| | sold under the reference KF6100 by the | |
| | company Shin-Etsu | |
| | Decamethyltetrasiloxane | 10.00 |
| | Cyclohexasiloxane | 26.00 |
| | Isododecane | 10.00 |
| | Dodecamethylpentasiloxane | 20.00 |
| B | Aqueous suspension of organopolysiloxane | 26.00 (16.38 AM) |
| | elastomer (63% elastomer) sold under the | |
| | reference BY 29-119 by the company Dow | |
| | Corning | |
| C | Mica-titanium oxide (63.5/36.5) sold under | 2.00 |
| | the reference EXP 1373-088C Sun Shine soft | |
| | fine red by the company Sun Chemical | |
| | TOTAL | 100 |

Procedure:

This composition is prepared by reproducing the preparation process described for Examples 19 and 20.

1. Composition, comprising a physiologically acceptable medium containing at least one oily phase, wherein said oily phase comprises:
(i) at least one silicone elastomer present within water droplets dispersed in said oily phase; and
(ii) at least one polyglycerylated silicone surfactant with an HLB greater than or equal to 8.

2. Composition according to claim 1, wherein the oily phase forms the external phase.

3. Composition according to claim 1, wherein the silicone elastomer (i) is in the form of particles of at least one partially or totally crosslinked organopolysiloxane elastomer.

4. Composition according to claim 1 wherein said silicone elastomer is present in said droplets in a form combined with at least one non-ionic surfactant.

5. Composition according to claim 1 wherein the silicone elastomer is present in the composition in an active material amount ranging from 0.1% to 30% by weight relative to the total weight of the composition.

6. Composition according to claim 1 wherein the surfactant in (ii) has the formula (I): 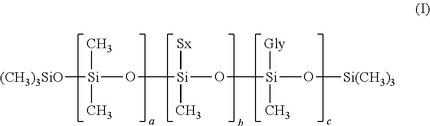
in which:
Sx is the radical —O(CH₃)₂SiO—Si(CH₃)₃,
Gly is the radical —C₃H₆O[CH₂—CH(OH)CH₂O]ₙH with n ranging from 1 to 5, and
a ranges from 1 to 1.4; b ranges from 0.02 to 0.04; and c ranges from 0.02 to 0.04.

7. Composition according to claim 1, containing from 1% to 15%, by weight of surfactant(s) (ii), relative to the total weight of the composition.

8. Composition according to claim 1 wherein the oily phase(s) comprise(s), or even consist(s) of, one or more silicone oil(s).

9. Composition according to claim 1 wherein the proportion of the oily phase(s) comprises from 35% to 95% by weight, of oil(s), relative to the total weight of the composition.

10. Composition according to claim 1 wherein the oily phase comprises at least one water-soluble compound, said water-soluble compound being carried within the water droplets of the aqueous dispersion of elastomer (i).

11. Composition according to claim 10, comprising water-soluble compounds having an amount of active material ranging from 0.0001% to 20% by weight, relative to the total weight of the composition.

12. (canceled)

13. Composition according to claim 1 wherein the oily phase comprises reflective particles of average size less than or equal to 30 μm, and made up of a natural or synthetic mica-based substrate coated with a single layer or several superimposed layers, the weight proportion of a TiO₂ layer, if present, remaining less than that of the mica.

14. (canceled)

15. (canceled)

16. (canceled)

17. Process for preparing a cosmetic composition according to claim 1 wherein said process, comprises at least one step of introducing a silicone elastomer in aqueous suspension into an oily phase and at least one step of introducing at least one polyglycerylated silicone surfactant with an HLB greater than or equal to 8 into said oily phase.

18. Process for preparing a cosmetic composition according to claim 2 wherein said process comprises at least the following two steps (a) and (b), in this order:
(a) providing an oily phase containing at least one polyglycerylated silicone surfactant with an HLB greater than or equal to 8; and
(b) introducing into said oily phase at least one silicone elastomer in aqueous suspension.

19. Process for preparing a cosmetic composition according to claim 10 wherein said process comprises at least:
a) a step of introducing at least one silicone elastomer in aqueous suspension into an oily phase, and
b) a step of introducing at least one polyglycerylated silicone surfactant with an HLB greater than or equal to 8 into said oily phase,
the two introducing steps being simultaneous or consecutive,
said aqueous suspension of silicone elastomer also containing at least one water-soluble compound.

20. Process for preparing a cosmetic composition according to claim 13 wherein said process comprises at least:
a) a step of introducing at least one silicone elastomer in aqueous suspension into an oily phase,
b) a step of introducing at least one polyglycerylated silicone surfactant with an HLB greater than or equal to 8 into said oily phase, and
c) a step of introducing the reflective particles into the oily phase containing said elastomer(s) and surfactant(s),
it being possible for steps (a) and (b) to be simultaneous or successive.

21. Process for making up and/or caring for a keratin material, wherein said process comprises a step of applying a composition according to claim 1.
